SEQUENCE LISTING (14 sequences)

SEQ ID NO: 1 - DNA, 5266 bp, Homo sapiens
ccgcctaatgagcgggcttttttttcttagggtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttcccctaccggtctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacccctgatcactgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcgtcgacatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagtaagatctttttccctctgccaaaaattatggggacatcatgaagccccttgagcatctgacttctggctaataaaggaaatttattttcattgcaatagtgtgttggaattttttgtgtctctcactcggaaggacataagggcggccgctagc

SEQ ID NO: 2 - DNA, 6162 bp, Homo sapiens
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacggtatcgataagcttgatatcgaattcatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactaggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 3 - protein, 722 aa, Homo sapiens
MEKNGNNRKLRVCVATCNRADYSKLAPIMFGIKTEPEFFELDVVVLGSHLIDDYGNTYRMIEQDDFDINTRLHTIVRGEDEAAMVESVGLALVKLPDVLNRLKPDIMIVHGDRFDALALATSAALMNIRILHIEGGEVSGTIDDSIRHAITKLAHYHVCCTRSAEQHLISMCEDHDRILLAGCPSYDKLLSAKNKDYMSIIRMWLGDDVKSKDYIVALQHPVTTDIKHSIKMFELTLDALISFNKRTLVLFPNIDAGSKEMVRVMRKKGIEHHPNFRAVKHVPFDQFIQLVAHAGCMIGNSSCGVREVGAFGTPVINLGTRQIGRETGENVLHVRDADTQDKILQALHLQFGKQYPCSKIYGDGNAVPRILKFLKSIDLQEPLQKKFCFPPVKENISQDIDHILETLSALAVDLGGTNLRVAIVSMKGEIVKKYTQFNPKTYEERINLILQMCVEAAAEAVKLNCRILGVGISTGGRVNPREGIVLHSTKLIQEWNSVDLRTPLSDTLHLPVWVDNDGNCAALAERKFGQGKGLENFVTLITGTGIGGGIIHQHELIHGSSFCAAELGHLVVSLDGPDCSCGSHGCIEAYASGMALQREAKKLHDEDLLLVEGMSVPKDEAVGALHLIQAAKLGNAKAQSILRTAGTALGLGVVNILHTMNPSLVILSGVLASHYIHIVKDVIRQQALSSVQDVDVVVSDLVDPALLGAASMVLDYTTRRIY

SEQ ID NO: 4 - protein, 400 aa, Homo sapiens
METYGYLQRESCFQGPHELYFKNLSKRNKQIMEKNGNNRKLRVCVATCNRADYSKLAPIMFGIKTEPEFFELDVVVLGSHLIDDYGNTYRMIEQDDFDINTRLHTIVRGEDEAAMVESVGLALVKLPDVLNRLKPDIMIVHGDRFDALALATSAALMNIRILHIEGGEVSGTIDDSIRHAITKLAHYHVCCTRSAEQHLISMCEDHDRILLAGCPSYDKLLSAKNKDYMSIIRMWLGDDVKSKDYIVALQHPVTTDIKHSIKMFELTLDALISFNKRTLVLFPNIDAGSKEMVRVMRKKGIEHHPNFRAVKHVPFDQFIQLVAHAGCMIGNSSCGVREVGAFGTPVINLGTRQIGRETGENVLHVRDADTQDKILQALHLQFGKQYPCSKIYGDGNAVPR

SEQ ID NO: 5 - protein, 369 aa, Homo sapiens
MEKNGNNRKLRVCVATCNRADYSKLAPIMFGIKTEPEFFELDVVVLGSHLIDDYGNTYRMIEQDDFDINTRLHTIVRGEDEAAMVESVGLALVKLPDVLNRLKPDIMIVHGDRFDALALATSAALMNIRILHIEGGEVSGTIDDSIRHAITKLAHYHVCCTRSAEQHLISMCEDHDRILLAGCPSYDKLLSAKNKDYMSIIRMWLGDDVKSKDYIVALQHPVTTDIKHSIKMFELTLDALISFNKRTLVLFPNIDAGSKEMVRVMRKKGIEHHPNFRAVKHVPFDQFIQLVAHAGCMIGNSSCGVREVGAFGTPVINLGTRQIGRETGENVLHVRDADTQDKILQALHLQFGKQYPCSKIYGDGNAVPR

SEQ ID NO: 6 - protein, 364 aa, Homo sapiens
MPIGDCSVAAKPRKQLLCSLFQTTLGYRARASGWKPMVICRGSHAFKDLINTYRMIEQDDFDINTRLHTIVRGEDEAAMVESVGLALVKLPDVLNRLKPDIMIVHGDRFDALALATSAALMNIRILHIEGGEVSGTIDDSIRHAITKLAHYHVCCTRSAEQHLISMCEDHDRILLAGCPSYDKLLSAKNKDYMSIIRMWLGDDVKSKDYIVALQHPVTTDIKHSIKMFELTLDALISFNKRTLVLFPNIDAGSKEMVRVMRKKGIEHHPNFRAVKHVPFDQFIQLVAHAGCMIGNSSCGVREVGAFGTPVINLGTRQIGRETGENVLHVRDADTQDKILQALHLQFGKQYPCSKIYGDGNAVPR

SEQ ID NO: 7 - protein, 293 aa, Homo sapiens
MEKNGNNRKLRVCVATCNRADYSKLAPIMFGIKTEPEFFELDVVVLGSHLIDDYGNTYRMIEQDDFDINTRLHTIVRGEDEAAMVESVGLALVKLPDVLNRLKPDIMIVHGDRFDALALATSAALMNIRNPILHIEGGEVSGTIDDSIRHAITKLAHYHVCCTRSAEQHLISMCEDHDRILLAGCPSYDKLLSAKNKDYMSIIRMWLGDDVNPKSKDYIVALQHPVTTDIKHSIKMFELTLDALISFNKRTLVLFPNIDAGSKEMVRVMRKKGIEHHPNFRAVKHVPFDQFIQ

SEQ ID NO: 8 - protein, 179 aa, Homo sapiens
MIEQDDFDINTRLHTIVRGEDEAAMVESVGLALVKLPDVLNRLKPDIMIVHGDRFDALALATSAALMNIRILHIEGGEVSGTIDDSIRHAITKLAHYHVCCTRSAEQHLISMCEDHDRILLAGCPSYDKLLSAKNKDYMSIIRMWLGSKEMVRVMRKKGIEHHPNFRAVKHVPFDQFIQ

SEQ ID NO: 9 - protein, 31 aa, Homo sapiens
TLVLFPNIDAGSKEMVRVMRKKGIEHHPNFR

SEQ ID NO: 10 - protein, 31 aa, Homo sapiens
TLVLFPNIDAGSKEMVQVMRKKGIEHHPNFR

SEQ ID NO: 11 - protein, 31 aa, Homo sapiens
TLVLFPNIDAGSKEMVWVMRKKGIEHHPNFR

SEQ ID NO: 12 - protein, 31 aa, Homo sapiens
TLVLFPNIDAGSKEMVLVMRKKGIEHHPNFR

SEQ ID NO: 13 - protein, 31 aa, Homo sapiens
TLVLFPNIDAGSKEMVRVMQKKGIEHHPNFR

SEQ ID NO: 14 - protein, 31 aa, Homo sapiens
TLVLFPNIDAGSKEMVRVMWKKGIEHHPNFR

(19) United States
(12) Patent Application Publication
(10) Pub. No.: US 2022/0088223 A1
(43) Pub. Date: Mar. 24, 2022

(54) METHODS AND COMPOSITIONS FOR INCREASING SIALIC ACID PRODUCTION AND TREATING SIALIC RELATED DISEASE CONDITIONS

(71) Applicant: Daniel Darvish, Chatsworth, CA (US)

(72) Inventors: Daniel Darvish, Los Angeles, CA (US); Yadira Valles-Ayoub, Los Angeles, CA (US)

(73) Assignee: Daniel Darvish, Chatsworth, CA (US)

(21) Appl. No.: 17/547,031

(22) Filed: Dec. 9, 2021

Related U.S. Application Data
(62) Division of application No. 16/006,767, filed on Jun. 12, 2018.
Application No. 16/006,767 is a continuation of application No. 14/285,602, filed on May 22, 2014, now Pat. No. 10,098,969.
Application No. 14/285,602 is a continuation of application No. 13/364,181, filed on Feb. 1, 2012, now abandoned.
(60) Provisional application No. 61/438,585, filed on Feb. 1, 2011.

(51) Int. Cl. A61K 48/00; C12N 15/52; A61P 21/00; C12N 15/85; A61K 38/52

(52) U.S. Cl. CPC A61K 48/005; C12N 15/52; C12Y 207/0106; C12N 15/85; A61K 38/52; A61P 21/00

(57) ABSTRACT

Disclosed herein are methods of expressing UDP-GlcNAc 2-Epimerase/ManNAc Kinase enzyme (GNE) peptide in a cell of a subject comprising: delivering into the cell of the subject an isolated nucleic acid expression construct that comprises a promoter operatively linked to a nucleic acid sequence encoding a GNE peptide or a therapeutically active fragment thereof, wherein the GNE peptide has the amino acid sequence of SEQ ID NO:3, wherein upon the delivering into the cell of the subject, the nucleic acid expression construct initiates expression of the GNE peptide or a therapeutically active fragment thereof. Also disclosed are methods of producing a GNE peptide in a cell comprising infecting the cell with an isolated nucleic acid construct that comprises a promoter operatively linked to a nucleic acid sequence encoding a GNE peptide or a therapeutically active fragment thereof, wherein the GNE peptide has the amino acid sequence of SEQ ID NO:3.

### CROSS-REFERENCE TO RELATED APPLICATIONS

This application is a divisional of U.S. patent application Ser. No. 16/006,767, filed Jun. 12, 2018 (pending), which is a continuation of U.S. patent application Ser. No. 14/285,602, filed May 22, 2014 (issued as U.S. Pat. No. 10,098,969 on Oct. 16, 2018), which is a continuation of U.S. patent application Ser. No. 13/364,181, filed Feb. 1, 2012 (abandoned), which claims priority to U.S. Provisional Application Ser. No. 61/438,585, filed Feb. 1, 2011 (expired), by Darvish et al., the disclosures of which are incorporated by reference herein, in their entireties for all purposes.

### FIELD OF THE INVENTION

The present invention is in the field of gene therapy methods and compositions for increasing production of sialic acid in a biological system by delivering the DNA coding region of the key enzyme of Sialic Acid biosynthesis (UDP-N-Acetylglucosamine 2-Epimerase/N-Acetylmannosamine Kinase, GNE)

### BACKGROUND OF THE DISCLOSURE

Hereditary Inclusion Body Myopathy (HIBM) is a young-adult onset progressive skeletal muscle wasting disorder, which causes severe physical incapacitation. There is currently no effective therapeutic treatment for HIBM. HIBM is an autosomal recessive disorder caused by mutation in the GNE gene. The GNE gene encodes for the bifunctional enzyme UDP-GIcNAc 2-epimerase/ManNAc kinase (GNE/MNK). This is the key rate-limiting enzyme catalyzing the first two reactions of cellular sialic acid production. Reduced sialic acid production consequently leads to decreased sialyation of a variety of glycoproteins, including critical muscle proteins such as α-dystroglycan (α-DG), neural cell adhesion molecule (NCAM), or neprilysin, or lead to altered expression of other genes such as ganlioside (GM3) synthase. This in turn leads to muscle degeneration. HIBM is also known as Distal Myopathy with Rimmed Vacuoles, Nonaka Myopathy, Vacuolar myopathy sparing the quadricepts, or GNE related myopathy.

### SUMMARY OF THE INVENTION

Disclosed herein are methods of expressing UDP-GlcNAc 2-Epimerase/ManNAc Kinase enzyme (GNE) peptide in a cell of a subject comprising: delivering into the cell of the subject an isolated nucleic acid expression construct that comprises a promoter operatively linked to a nucleic acid sequence encoding a GNE peptide or a therapeutically active fragment thereof, wherein the GNE peptide has the amino acid sequence of SEQ ID NO:3, wherein upon the delivering into the cell of the subject, the nucleic acid expression construct initiates expression of the GNE peptide or a therapeutically active fragment thereof.

Also disclosed are methods of delivering an encoded GNE enzyme comprising: a) creating an intravenous access at a point below a knee or an elbow of a limb of a subject; b) applying a tourniquet at a point proximal to the rest of the body of the subject than the intravenous access point; c) introducing a single dose of an isolated nucleic acid expression construct into the limb through the intravenous access, wherein the single dose is of sufficient volume to increase intravascular pressure for extravasation of the polynucleotide; wherein, the isolated nucleic acid construct comprises a promoter operatively linked to a nucleic acid sequence encoding a GNE peptide or a therapeutically active fragment thereof, wherein the GNE peptide has the amino acid sequence of SEQ ID NO:3.

Further, disclosed are methods of producing a GNE peptide in a cell comprising infecting the cell with an isolated nucleic acid construct that comprises a promoter operatively linked to a nucleic acid sequence encoding a GNE peptide or a therapeutically active fragment thereof, wherein the GNE peptide has the amino acid sequence of SEQ ID NO:3.

### BRIEF DESCRIPTION OF THE DRAWINGS

FIG. 1 is a diagram of the NTC8685-GNE expression vector described herein.

FIG. 2 shows the nucleotide sequence of NTC8685-GNE vector (SEQ ID NO:1).

FIG. 3 is a diagram of UMVC3-GNE vector.

FIG. 4 shows the nucleotide sequence of UMVC3-GNE vector (SEQ ID NO:2).

FIG. 5 shows the amino acid sequence of GNE protein enzyme (SEQ ID NO:3).

FIG. 6 shows the amino acid sequence of GNE isoforms and Allosteric domain. Common allosteric domain mutations allowing higher Sialic Acid production are illustrated (R263Q/W/L, and R266Q/W).

FIG. 7 is a bar graph of sialic acid production in GNE-null CHO cells. In comparison to untreated cells (“Media”, “Empty Vector”), sialic acid production was significant greater in cells transfected with GNE plasmids.

FIG. 8 is a bar graph of Sialic Acid GNE-null CHO cells, comparison of UMVC3 and NTC8685 Vectors.

FIG. 9 is a bar graph of Sialic Acid content cell fractions of GNE-null CHO cells, comparison of UMVC3 and NTC8685 Vectors.

FIG. 10 is a bar graph showing the relative in-vitro dose comparison of GNE vector vs ManNAc

### DETAILED DESCRIPTION OF THE EMBODIMENTS

Disclosed herein are gene therapy methods and compositions for increasing production of sialic acid in a biological system by delivering the DNA coding region of the key enzyme of Sialic Acid biosynthesis (UDP-N-Acetylglucosamine 2-Epimerase/N-Acetylmannosamine Kinase, GNE). Disease conditions that will benefit from increased cellular sialic production, or enhanced GNE functions, include, but not limited to, Hereditary Inclusion Body Myopathy (HIBM) or Distal Myopathy with Rimmed Vacuoles (DMRV). The present methods and compositions also relate to reducing or eliminating non-human sialic acids (e.g. N-Glycolylneuraminate, Neu5Gc) from human cells or tissues. Non-human sialic acids may contribute to various human diseases, and long term reduction of cellular levels of non-human sialic acid may prove beneficial in preventing and treating those disease processes (WO/2010/030666) (Varki 2009). Increasing cellular production of Acetylneuraminate (Neu5Ac) can reduce cellular content of non-human sialic acids.

Being personally affected by HIBM, the inventor has developed and validated a gene therapy vector (plasmid, naked polynucleic acid) through in-vitro studies over the past 7 years. Through many years of medical literature searches, and evaluation of the data regarding various in-vivo delivery methods and vectors, an elegant and facile delivery method was chosen using a variation of a procedure known as the “Bier Block”. Bier Block has been used safely in medical practice for over 100 years (dos Reis 2008).

As described below, the combination of the specific disease processes, the plasmids, and delivery method has numerous advantages over any others described to date. These advantages allow for facile translation for practical use in human and animal models.

Disclosed herein are the components of pharmacologic products and methods of delivering the pharmacologic products to the skeletal muscles or other organs (e.g. liver) of animals or human patient (e.g. patient affected with HIBM). The pharmacologic products can be polynucleotides encoding the unmodified or modified forms of GNE protein, polypeptides or amino acid sequences and/or or recombinant proteins, polypeptides or amino acid sequences encoded by the unmodified or modified forms of GNE nucleotide. In some embodiments, the delivery methods include (1) external or internal occlusion of major vessels (arteries, veins, and/or lymphatic system) to achieve vascular isolation of the target organ systems, group of organs/tissues, or body area, and (2) administration of the therapeutic product using vascular (e.g. intravenous) access. In some embodiments, the body organs/tissues/area that are isolated (target organs) are exposed to the compound being delivered, while in other embodiments, the body organs/tissues/area are protected from such exposure.

### Description and Improvements of the Therapeutic Gene (GNE)

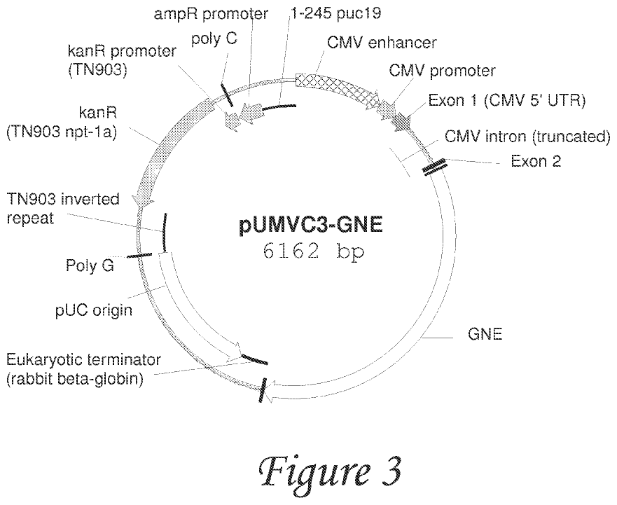

In some embodiments, the therapeutic products disclosed herein are polynucleotide (DNA) molecules, while in other embodiments, they are polypeptide (protein, protein fragments, amino acid sequences) molecules. In some embodiments, the polynucleotide molecule, either linear or circular, may contain various elements in addition to the coding sequence that encodes for the GNE protein, or a modified form of the GNE protein, that is or becomes biologically active within a biological system. GNE protein has the sequence (FIG. 3).

In some embodiments, the therapeutic methods disclosed herein are commonly known as “Gene Therapy”, and comprise the administration of the above polynucleotide molecule. In other embodiments, the therapeutic methods disclosed herein are commonly known as “Enzyme Replacement Therapy (ERT)”, and comprise the administration of the GNE protein, or a modified form of the GNE protein, that is or becomes biologically active within a biological system.

GNE encodes for the key enzyme of sialic acid production (UDP-N-Acetylglucosamine 2-epimerase/N-Acetylmannosamine Kinase). Several disease conditions can benefit from increased expression of GNE. The most notable being the severely debilitating progressive muscle wasting disorder known as GNE related myopathy, Hereditary Inclusion Body Myopathy (HIBM) and one of its distinct forms known as IBM2, or Distal Myopathy with Rimmed Vacuoles (DMRV)

The GNE enzyme components or domains (e.g. series of 10 or more sequential amino acids) may be recombined to enhance desired functions of the GNE gene and reduce or eliminate undesired functions. For example, if production of high amounts of sialic acid (NeuAc) is desired in biological organisms, for example prokaryotes or eukaryotes, one may optimize the epimerase domain of the GNE gene to eliminate or reduce the allosteric inhibitory domain function. In organisms and animals having redundant ManNAc kinase activity, such as other enzymes able to efficiently perform phosphorylation of ManNAc, one may also reduce or eliminate the GNE kinase domain to reduce the size, the minimum effective dose, and/or maximize the maximum tolerable dose in a biological system.

Although the GNE enzyme, or various components or domains thereof, is also known to have cellular functions besides production of sialic acid (Hinderlich, Salama et al. 2004; Broccolini, Gliubizzi et al. 2005; Krause, Hinderlich et al. 2005; Salama, Hinderlich et al. 2005; Penner, Mantey et al. 2006; Wang, Sun et al. 2006; Amsili, Shlomai et al. 2007; Amsili, Zer et al. 2008; Kontou, Weidemann et al. 2008; Kontou, Weidemann et al. 2009; Paccalet, Coulombe et al. 2010), hyposialylation of critical cellular molecules play an important role in human disease process (Huizing, Rakocevic et al. 2004; Noguchi, Keira et al. 2004; Saito, Tomimitsu et al. 2004; Tajima, Uyama et al. 2005; Ricci, Broccolini et al. 2006; Galeano, Klootwijk et al. 2007; Sparks, Rakocevic et al. 2007; Nemunaitis, Maples et al. 2010).

Increasing sialic acid and NeuAc/NeuGc ratio in biological systems is desired for several known reasons in human subjects. Mammals produce two different sialic molecules: (1) N-Acetylneuraminic acid (NANA or Neu5Ac), and (2) N-Glycolylneuraminic acid (Neu5Gc). CMP-NANA is converted to CMP-Neu5Gc by CMP-NANA hydoxylase (CMAH). Unlike other primates and mammals (including cow), humans are genetically deficient in Neu5Gc due to an Alu-mediated inactivating mutation of CMAH (Chou, Hayakawa et al. 2002). Thus, Neu5Ac is the only sialic acid produced by humans and many humans produce antibodies against Neu5Gc (Tangvoranuntakul, Gagneux et al. 2003). The NeuGc found in human tissues and cells are believed to be from food or cell culture media. Humans produce antibodies against NeuGc, potentially contributing to chronic inflammation, and various common disorders in which chronic inflammation is believed to be a significant factor (e.g. cancer, atherosclerosis, autoimmune disorders) (Hedlund, Padler-Karavani et al. 2008; Varki 2009). NeuGc can also promote human diseases, such as hemolytic uremic syndrome (HUS). A major cause of HUS is Shiga toxigenic Escherichia coli (STEC) infection. A highly toxic Shiga toxin subtilase cytotoxin (SubAB) prefers binding to glycan terminating in NeuGc (Lofling, Paton et al. 2009). This information increases our concern that NeuGc may also increase human susceptibility to some infectious agents.

Thus, it is desired to increase the content of NeuAc (human sialic acid) in food, and reduce the proportion of NeuGc found in meat and milk products. A potentially effective method to accomplish this is to increase GNE expression, and reduce or eliminate the CMAH expression in biological systems or organism used as either human or animal food (e.g. milk, meat, diary, and other animal based products). CMAH may be reduced by either of genetic or metabolic technologies, including, but not limited to, genetic modification of animals to produce CMAH knock-out or knock-down animals, reduction of CMAH enzyme expression by polynucleotide technologies (expressed as inhibitory RNA or antisense oligonucleotide), or inhibition of CMAH enzyme by metabolic substrate analogues. NeuGc may also be reduced in biological systems by overexpression of the enzyme that converts NeuGc to NeuAc.

With few exceptions, plants do not typically produce sialic acid. GNE and other sialic acid pathway enzymes can be used in plant, vegetable, and fruit crops to increase sialic acid in food.

Modifications, additions, and/or removal of polynucleotide elements (e.g. promoters, enhancers, repeat elements) can be used to enhance expression in various tissues/organs or developmental stages, which may be desired in various fields of biotechnology including, but not limited to, pharmacologic, food, and cosmetic industries.

Because skeletal muscle is an important tissue that is readily accessible and that is highly vascularized, it could be used as a factory to produce proteins with therapeutic values (reviewed in (Lu, Bou-Gharios et al. 2003; Ratanamart and Shaw 2006)). Indeed, it has been demonstrated that functional therapeutic proteins can be synthesized by the skeletal muscle and secreted into the blood circulation in sufficient amount to mitigate the pathology associated with disorders such as hemophilia, Pompe disease, Fabry's disease, anaemia, emphysema, and familial hypercholesterolemia. The ability to express recombinant proteins in skeletal muscle is also an important issue for the treatment of neuromuscular disorders such as Duchenne and limb girdle muscular dystrophy. These disorders are caused by mutations of a gene that produces an essential muscle protein One potential treatment for such disorders is gene transfer, whose objective is to introduce into the muscle a normal and functional copy of the gene that is mutated.

Thus, in one aspect, disclosed herein are methods to utilize muscle as protein factory to over-produce and secrete sialic acid. In some embodiments, the methods disclosed herein result in an increase of Neu5Ac biosynthesis in plasma, and the reduction of Neu5Gc concentration from cells.

### Description and Improvement of the Therapeutic Product

In some embodiments, the therapeutic product is a polynucleotide, while in other embodiments, the therapeutic product is a polypeptide. In some embodiments, the polynucleotide is a DNA molecule, which can comprise the full-length coding region for a protein, the coding region for a domain of a protein, or a coding region for a protein fragment, which is shorter than a recognized and identified domain of a protein. Thus, the polynucleotides disclosed herein can range from oligomers of at least 15 base pairs in length to DNA molecule comprising the full-length coding region for a protein.

In some embodiments, the polypeptide is a full-length protein, e.g., an enzyme or a receptor, while in other embodiments, the polypeptide is a protein fragment. In some embodiments, the protein fragment corresponds to a recognized and identified domain of a full-length protein, while in other embodiments, the polypeptide is shorter than a recognized and identified domain of a protein. Thus, the polypeptides disclosed herein can range from oligomers of at least 5 amino acids in length to full-length proteins. In some embodiments, the protein fragment is a therapeutically active protein fragment. By “therapeutically active protein fragment” it is meant that the protein fragment under physiological conditions has the same biochemical activity (e.g., catalyzes the same reaction) as the wild-type GNE protein, although it may perform the function at a different rate.

In some embodiments, the polynucleotide is a linear DNA molecule whereas in other embodiments, the polynucleotide is a circular DNA molecule.

In some embodiments, the polynucleotide is a circular DNA (plasmid, miniplasmid, or minicircle) able to express the GNE gene in the desired biological system. The NTC8685 vector described in this application has few benefits, which include reduced size, reduced bacterial sequence content, and antibiotic free selection. Other vectors known to those of skill in the art can also be used with the methods described herein.

In some embodiments, the polynucleotide therapeutic product, whether linear or circular, is administered as naked DNA, combined with other molecules to produce various cationic or anaionic particles, or co-administered with other pharmacological agents (e.g. exipients, vasodialaters, analgesics, etc,) to maximize efficacy of therapy and minimize patient discomfort. Instead of a polynucleotide, other pharmacologic products may be administered using the stated delivery method.

Unlike in vitro studies, where net positive zeta potential is a more efficient cellular entry of a polyneuleotide, in vivo transduction of skeletal muscle seems to be more efficient using a polynucleotide having a net negative charge (PCT WO/2004/062368).

In one embodiment, muscle specific promoters may be used to reduce chance of host immune response against the transgene and enhance the duration of intramuscular expression of the transgene. The backbone plasmid elements can be altered to allow for muscle specific expression. The ability to achieve high-level and long-term recombinant protein expression after gene transfer in skeletal muscle is desired in many disease conditions. This can be achieved using promoters and enhancers specific for muscle.

Several different muscle specific promoters have been described to date. The muscle creatine kinase (MCK) promoter and truncated versions are the most common muscle specific promoters used (Hauser, Robinson et al. 2000; Yuasa, Sakamoto et al. 2002; Sun, Zhang et al. 2005; Sebestyen, Hegge et al. 2007; Wang, Li et al. 2008). The synthetic C5-12 promoter and similar promoters show promise of being muscle specific while driving high expression of transgene (Li, Eastman et al. 1999). This C5-12 promoter drives expression levels similar to the ubiquitous CMV promoters in AAV vectors (Gonin, Arandel et al. 2005). The C5-12 can be further improved by adding the MCK enhancer (E-Syn promoter) (Wang, Li et al. 2008). The hybrid α-myosin heavy chain enhancer-/MCK enhancer-promoter (MHCK7) promoter also was used for high expression in muscles (Salva, Himeda et al. 2007). The desmin promoter is also recently described as a muscle-specific promoter capable or driving high level expression in muscle cells (Pacak, Sakai et al. 2008; Talbot, Waddington et al. 2010). The upstream enhancer elements (USE, USEx3/ΔUSEx3) of genes such as the troponin gene is also a promising candidate for developing muscle specific promoters (WO 2008124934 20081023; Blain, Zeng et al. 2010).

As disclosed herein, the GNE-encoding sequences, and/or the associated delivery vehicles used therewith, may be targeted towards specific cell types, for example, muscle cells, muscle tissue, and the like. For example, the promoter associated with the GNE coding sequence can be made to express GNE only in specific tissues or developmental stages. Alternatively, the expression cassette can be packaged with other molecules, compounds, or biologic moieties (e.g. protein/carbohydrate/lipid containing molecules, part or whole antibody molecules, part or whole cytokine molecules, viral capsids) to generate a biological mixture or specific biological particles designed to bind to and enter specific cell types. This binding or affinity can facilitate the uptake of the DNA into the cell. For delivery into muscle, in particular, anionic, non-liposomal, DNA containing particles are well-suited. However, cationic (liposomal) as well as other DNA containing biological mixtures or particles are also suited for uptake into myopathic muscle with compromised cell wall. In some embodiments, these protein, carbohydrate, and/or lipd containing molecules targeting moieties are, but are not limited to, microbial, plant, microbial, or synthetic compounds (e.g. antibodies, cytokines, lectins, other large or small molecules).

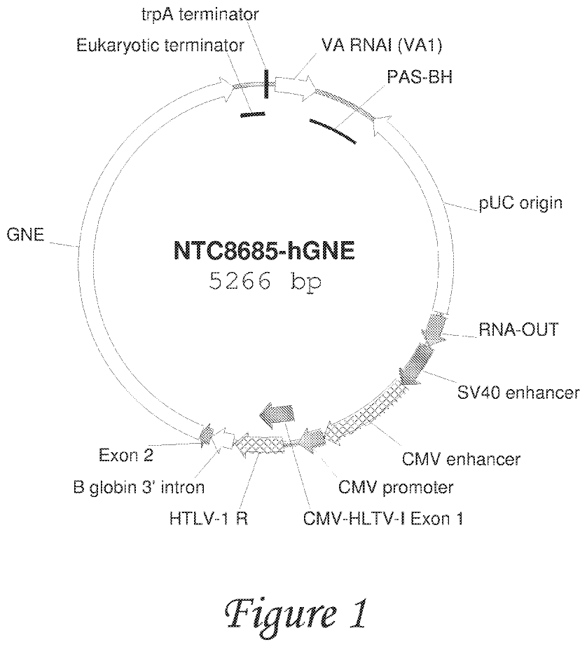

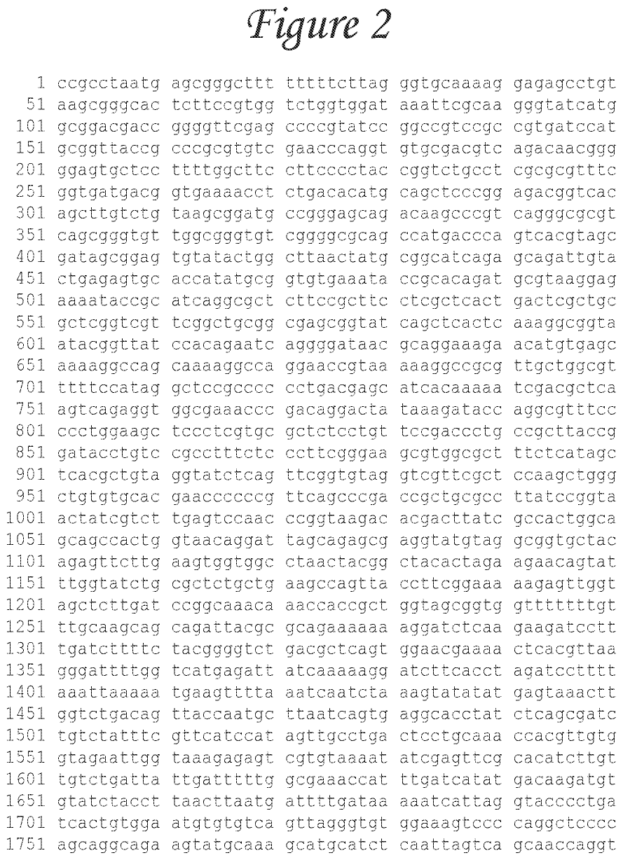

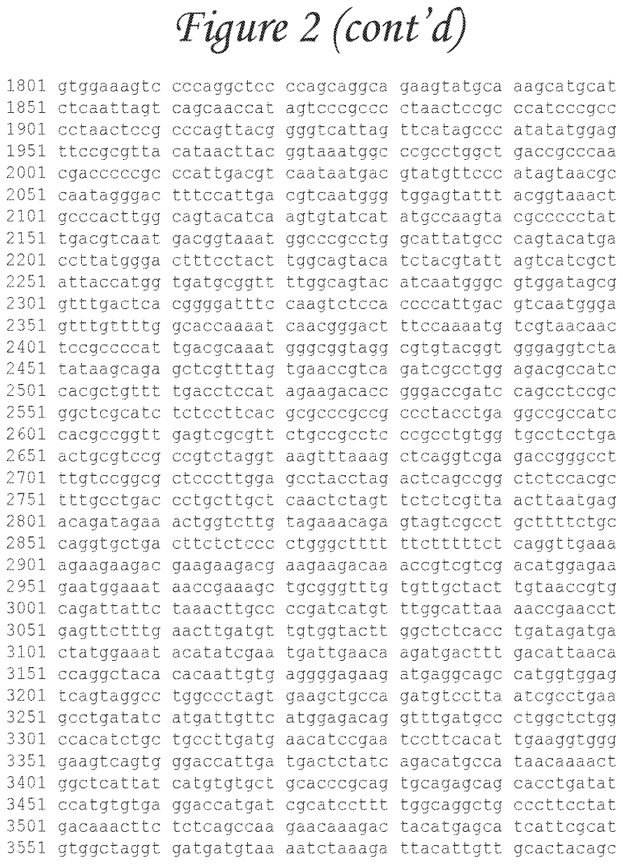

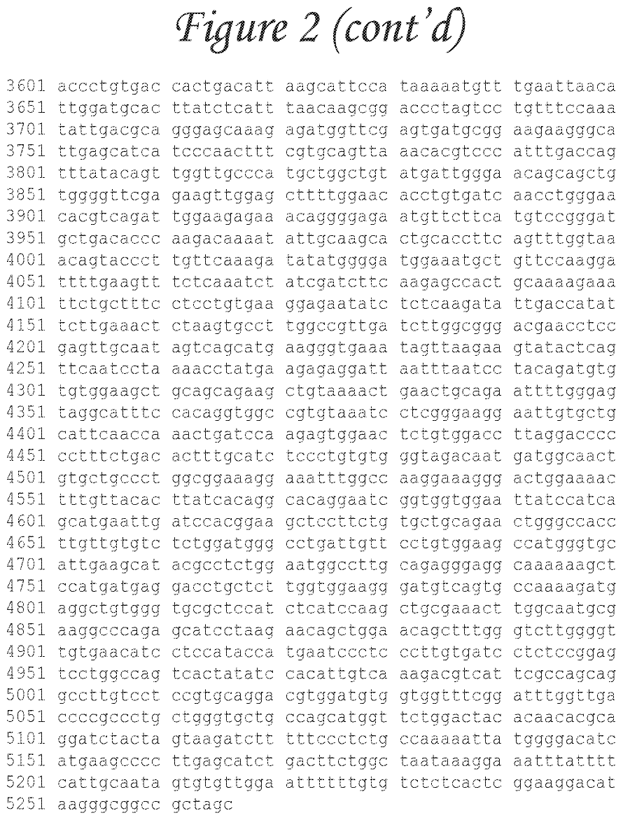

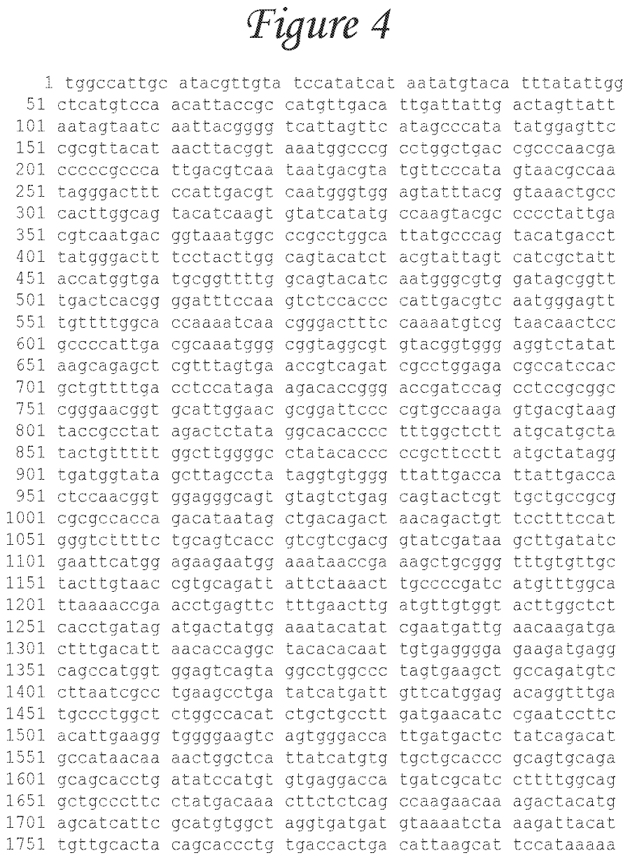

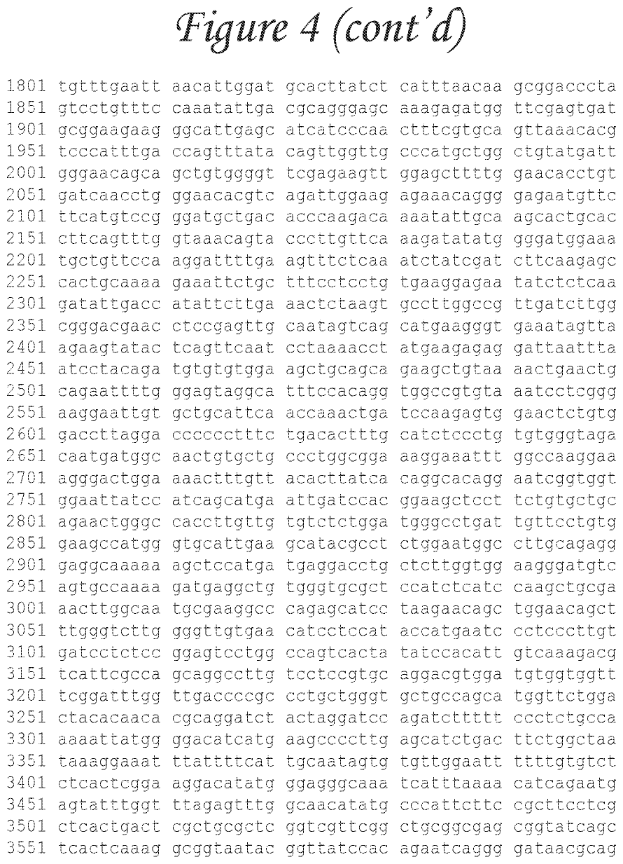

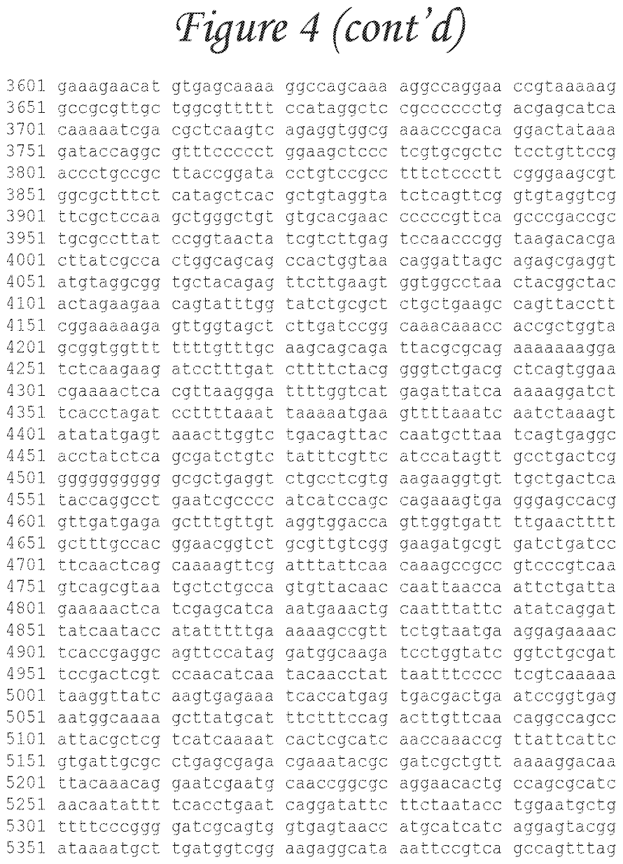

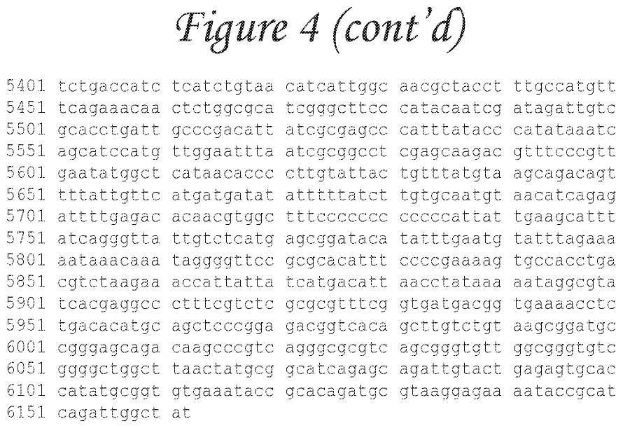

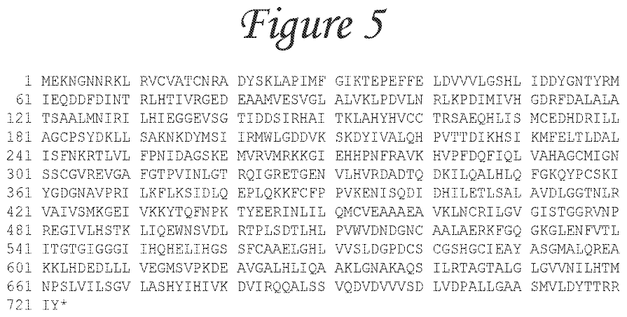

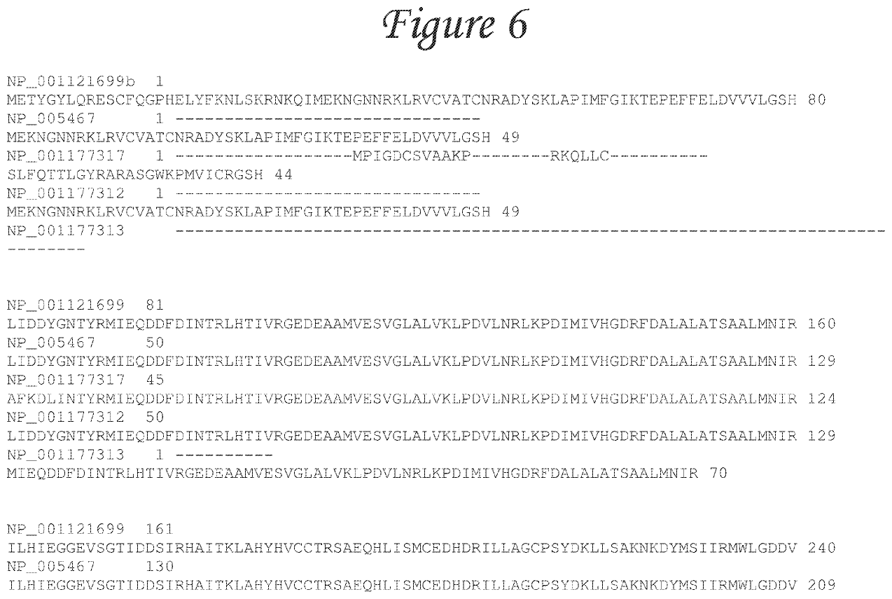

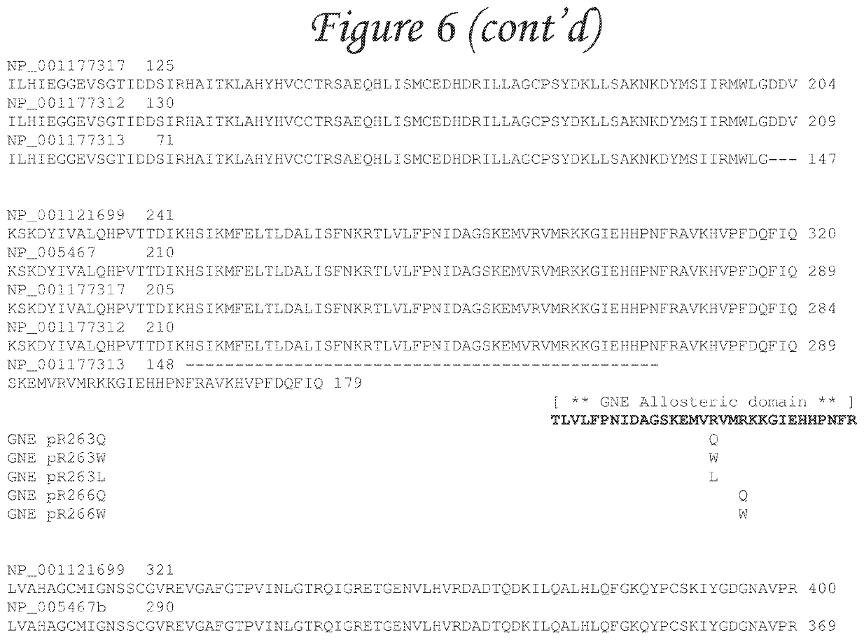

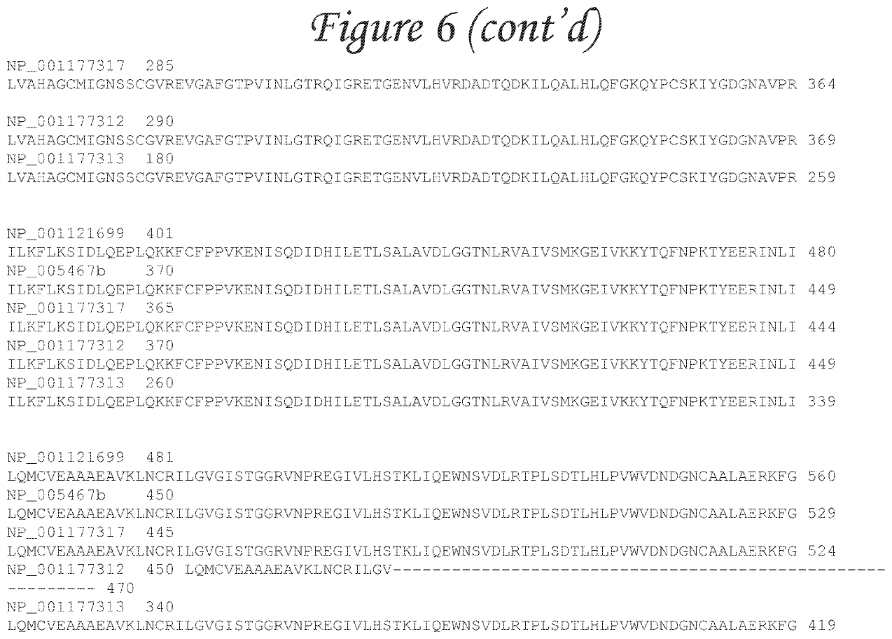

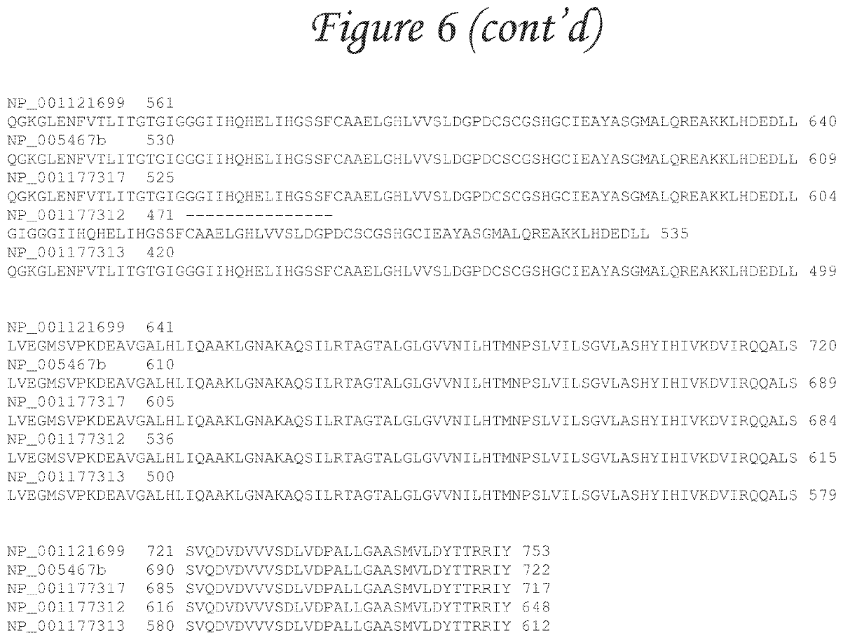

In some embodiments, polynucleotides products described herein comprise the following elements: 1) Bacterial Control Elements, which are active in bacteria for the purpose of selection and growth process, 2) Eukaryotic Control Elements, which are active in eukaryotic or mammalian cells for the purpose of expression of a therapeutic gene product or recombinant protein, and 3) the GNE coding region, which is the therapeutic gene product or recombinant gene. In some embodiments, prokaryotic/bacterial selection marker is based on antibiotic resistance (e.g. kanamycin resistance, as present in the UMVC3 vector, FIG. 3), or RNA based (e.g. RNA-OUT, present on the NTC8685 vector, FIG. 1). In other embodiments, other elements are used for efficient plasmid production (e.g. pUC orgin depicted in both UMVC3, FIG. 3, and NTC8684, FIG. 1) The nucleotide sequence of NTC8685-GNE vector is set forth in FIG. 2 and in SEQ ID NO:1, while the nucleotide sequence of UMVC3-GNE vector is set forth in FIG. 4. In additional embodiments, eukaryotic promoter, enhancer, introns or other elements are used for efficient transcription and translation of the therapeutic protein encoded by the GNE gene

To minimize potential spread of antibiotic resistance, prokaryotic selection marker that is not based on antibiotic resistance is preferred by regulatory agencies such as World Heath Organization (WHO), US Food and Drug Administration (FDA), or European Agency for the Evaluation of Medicinal Products (EMEA) (Williams, Carnes et al. 2009).

Rationale for using plasmid DNA: Clinical use of naked or plasmid DNA (pDNA) to express therapeutic genes is a promising approach to treat muscle disease caused by IBM2. Naked DNA as gene therapy vehicle has an excellent safety record and repeat administration in the same subject can achieve higher expression levels. (Hagstrom, Hegge et al. 2004; Wolff, Lewis et al. 2005; Wolff, Budker et al. 2005; Herweijer and Wolff 2007; Braun 2008; Duan 2008; Zhang, Wooddell et al. 2009) Depending on method of delivery, pDNA delivered to skeletal muscle of rodents or primates is retained in myofibers and expresses the encoded gene product for many months (Danko, Fritz et al. 1993; Danko, Williams et al. 1997; Sebestyen, Hegge et al. 2007). Unlike Adeno-Associated Virus (AAV) and other viral vectors which can induce cellular or humoral immunity(Yuasa, Yoshimura et al. 2007; Mingozzi, Meulenberg et al. 2009), pDNA does not typically elicit an immune response against the vector (Hagstrom, Hegge et al. 2004; Romero, Braun et al. 2004; Glover, Lipps et al. 2005; Wolff, Budker et al. 2005), which makes it possible to repeat administrations in same subject. Additionally, compared to viral or based vectors, pDNA is relatively inexpensive to produce in large quantities and remains stable for many months (Walther, Stein et al. 2003; Urthaler, Ascher et al. 2007; Voss 2007).

### Method of Delivery. Description and Improvement of the Delivery Method

In one embodiment of the hydrodynamic infusion, an external tourniquet is placed on the limb of a human being or animal, and the therapeutic product is administered using a peripheral intravenous access using a specific volume (typically 30-50% of the limb volume below the tourniquet) in a specific amount of time or volume flow (typically 1-3 ml/second). This is very similar to commonly used medical procedures known as the “Bier Block”, which has been used safely and effectively for more than a century to reduce the exposure and dose of pharmacologic compounds. Bier Block has been used to induce intravenous regional anesthesia (eliminating the need for general anesthesia) in arm or hand surgery (dos Reis 2008; Vlassakov and Bhavani 2010). Similar method is used in oncology by the name of “isolated limb infusion” for the administration of chemotherapeutic compounds to a specific limb, allowing for reduction in dose and exposure to internal organs (Kroon and Thompson 2009). Placing a tourniquet on limbs has also been used effectively for many centuries to reduce bleeding following severe trauma, or to reduce exposure of internal organs to toxins following exposure (e.g. venomous snake and other animal bites).

When administering gene therapy or biologics using the same or very similar delivery, the delivery method is described in medical literature by multiple names, including “hydrodynamic”, “transvenular”, “transvenous”, “transvascular”, “vascular”, “retrograde”, “limb vein”, “peripheral vein”, “intravenous”, “intravascular”, “retrograde”, “extravasation”, “high pressure”, “pressurized”, “isolated limb”, “vascular isolation”, “vascular occlusion”, “blood flow occlusion”, or any combination thereof (Su, Gopal et al. 2005; Sebestyen, Hegge et al. 2007; Vigen, Hegge et al. 2007; Zhang, Wooddell et al. 2009; Haurigot, Mingozzi et al. 2010; Hegge, Wooddell et al. 2010; Powers, Fan et al. 2010). Despite specific concerns, post-phlebitic syndrome or post-procedure angiopathy has not been noted following performance of vascular occlusion procedures following canine (dog) studies (Haurigot, Mingozzi et al. 2010).

In some embodiments, disclosed herein, the delivery method has been improved. Human and animal limbs of same volume may be composed of varying ratios of muscle and non-muscle (e.g. fatty or scar) tissues. Muscle is often more vascular and requires higher blood flow that lipid or scar tissue. Thus, administering therapeutic products using a specific volume may not confer optimum distribution of the therapeutic product in limbs of individuals. Limbs with higher muscle/non-muscle tissue may require higher infusion volumes to achieve same therapeutic benefit. Controlling the infusion based on intravascular (or infusion line) pressure and duration of infusion may convey improved distribution of therapeutic product to the target limb. The following alterations of the described method accordingly improve this delivery method:
1) Placing the tourniquet of specific pressure roughly 2-4× the systolic pressure (e.g. 320 mmHg for a human patient).
2) Rapid increase of flow to achieve a specific intravascular (or infusion line) pressure typically below the tourniquet pressure (e.g. if tourniquet pressure is maintained at 320 mmHg, the infusion line pressure maintained 280-300 mmHg)
3) Maintaining the infusion line pressure by controlling infusion flow rate.
4) Maintaining the infusion line pressure for a specific duration of time (15 minutes).
5) Using a specifically designed device to safely achieve parameters described above in 1 and 2. Such device may automatically control the flow rate and pressure of the infusion line based on the set tourniquet pressure. For safetly, such device would automatically stop infusion (flow rate of zero mL/sec) upon detection of parameters such as sudden drop in infusion line pressure, air bubble within the infusion line, or fluid level within the container holding the fluid to be infused.

By selecting the site of vascular administration distal or proximal to the site of vascular occlusion, one can either expose or protect the target organs, tissues, or body area.

Rationale for using HLV delivery method: Although commonly used for DNA vaccination trials, pDNA delivered by instramuscular (IM) approach is inefficient for muscle diseases demanding delivery of therapeutic product to an entire limb or the whole body (Jiao, Williams et al. 1992). Intravenous (IV) plasmid is cleared rapidly by the liver (Liu, Shollenberger et al. 2007). However, combined with hydrodynamic limb vein (HLV) delivery, pDNA administered IV can effectively and uniformly transfect skeletal muscle of an entire limb in small and large animals including non-human primates (Hagstrom, Hegge et al. 2004), that results in reversible microvasculature damage (Toumi, Hegge et al. 2006; Vigen, Hegge et al. 2007). A single dose can result in long-term gene expression, and the ease of repeat administration makes HLV suitable for delivering GNE transgene to the limbs of IBM2 patients. Using a tourniquet, blood flow in an arm or leg temporarily occluded, and a plasmid DNA solution is rapidly injected intravenously. This elevates the pressure within the occluded region, leading to remarkably efficient migration of the gene vehicle into the adjoining myofibers. Blood flow is restored to normal in 10-20 minutes, with no irreversible or persistent adverse affects. Similar high pressure intravenous approaches are being adopted and adapted for delivery of DNA, and possibly other potential therapeutic molecules, to various organs. (Al-Dosari, Knapp et al. 2005; Arruda, Stedman et al. 2005; Wolff, Lewis et al. 2005; Herweijer and Wolff 2007; Toromanoff, Cherel et al. 2008).

IBM2/DMRV is an ideal orphan disorder to be treated by pDNA gene delivery using HLV for the following reasons:

Low GNE expression may be therapeutic: GNE gene is relatively small (cDNA size 2,169 bp, coding for 722 amino acids), functioning as a protein enzyme that is expressed at low levels in skeletal muscle. Expression of low amounts of wild-type, or very low amounts of sialuria form of GNE, may prove remarkably effective or even curative. Additionally, it is possible to use the hypermorphic (Sialuria) form of the GNE gene allowing for very low expressions of the GNE gene to translate to significant therapeutic benefit. This is in sharp contrast to other muscle diseases such as Duchenne' or Becker muscular dystrophies where relatively large amounts of dystrophin (or truncated mini-dystrohpin) are needed to realize therapeutic benefit.

Treating limbs alone may be sufficient therapy: IBM2 notably affects muscles of arms and legs. Trunk muscles are clinically affected later in disease course. Vital organs, including heart and lungs, are not clinically affected in vast majority of patients. By saving arm and leg function, we can significantly improve quality of life and delay loss of independence.

Host immune response to the transgene is unlikely: Over 99% of known patients express GNE protein that differs from wild-type by one amino acid (missense mutation). Additionally, GNE is evolutionarily conserved with 98% homology between mice and men at the amino acid level. Thus, the chance of host immune response or producing neutralizing antibodies against the GNE transgene is minimal. Coupling GNE with a muscle specific promoter such as creatine kinase (CK) further reduces chance of host antibody response (Fabre, Bigey et al. 2006).

Potential for beneficial bystander or distant effects: Unlike dystrophinopathies, where expression of dystrophin (large structural protein) within a myofiber seems to benefit only the site of injection, in IBM2 it is likely that Neu5Ac (small molecule, 9 carbon sugar) will not remain within a limited region of the myofiber. Neu5Ac produced by one myofiber may benefit neighboring myofibers, and ManNAc or Neu5Ac in serum may benefit the myofibers exposed to that serum. Following data further support this hypothesis: (a) Sia deficient mouse models are able to use Neu5Ac present in serum (Malicdan, Noguchi et al. 2009) (b) hyposialylated cells became re-sialylated after their growth medium was supplemented with ManNAc (Schwarzkopf, Knobeloch et al. 2002) and (c) adding 5 mM ManNAc or Neu5Ac, but not GlcNAc, to the media restored the sialic acid content of primary DMRV fibroblasts or myotubes from 60-75% of control to normal levels (Noguchi, Keira et al. 2004). Bystander effect, and possibility of distant effect, was observed in a recent single patient trial (Nemunaitis, Maples et al. 2010). The patient received GNE-lipoplex intramuscular injection of forearm (Extensor Carpi Radialis Longus, ECRL). Transient increase in strength, recombinant GNE (rGNE) expression, and increase of cell surface sialic acid was observed at the injection site and adjacent compartment muscles. Possibility of distant effect was also suggested following the surprising observation that distant muscle groups (trapezius and quadriceps) improved transiently in correlation with left ECRL rGNE transgene expression and increased sialylation (Nemunaitis, Maples et al. 2010).

### Safety/Toxicology

Based on available information, GNE plasmid is expected to be a very safe vector for use in IBM2 patients. Generally, naked DNA as gene therapy vehicle has an excellent safety record and repeat administration in the same subject can achieve higher expression levels. (Hagstrom, Hegge et al. 2004; Wolff, Lewis et al. 2005; Wolff, Budker et al. 2005; Herweijer and Wolff 2007; Braun 2008; Duan 2008; Zhang, Wooddell et al. 2009).

Safety of GNE plasmid: Rodent toxicology studies using GNE-plasmid are currently underway. Preliminary data suggests naked plasmid will prove much safer than GNE-lipoplex that has already been administered to a human patient (Phadke, Jay et al. 2009; Nemunaitis, Maples et al. 2010). We conducted a recent pre-GLP toxicology study of 14 day duration on 12 mice (strain B6; FBV mixed inbred, 6 male and 6 female of age 4-10 months). Male and female mice were divided equally and randomly into experiment and control groups. The experiment group received high dose GNE plasmid (0.6 mg suspended in 0.1 ml normal saline) administered via IV tail, and the control group received only 0.1 ml normal saline. The groups were further divided into 3 dose frequency groups of 2 mice (1 female, 1 male) each as follows: 1) every day administration for 14 days, 2) every other day administration, and 3) once per week. All animals survived the experiment. No significant change were observed between the experiment and the control groups with respect to all measured parameters, which included body weights, temperature, food and water intake, CBC blood tests (performed at pre-dose day 1 and at necropsy on day 15). No significant change in the gross pathology was observed between the experiment and the control groups with respect to 12 organs, including brain, lung, heart, liver, kidney, spleen, stomach, intestines, bladder, genitals, lymph nodes, and muscle. The daily human equivalent dose (HED) was 120 mg, and the maximum 14 day total HED was 1440 mg.

Safety of GNE-lipoplex: In comparison to naked plasmid GNE, the GNE-lipoplex form is more toxic. To produce the lipoplex, the plasmid vector was encapsulated in a cationic liposome composed of 1,2-dioleoyl-3-trimethylammonium-propane (DOTAP) and cholesterol (GNE-lipoplex). The vector was injected into BALB/c mice, and ingle intravenous (IV) infusion of GNE-lipoplex was lethal in 33% of animals at 100 μg (0.1 mg) dose, with a small proportion of animals in the 40 μg cohort demonstrating transient toxicity (Phadke, Jay et al. 2009). Based on a poster presented at 2010 ASGCT conference (Phadke, Jay et al. 2010), the maximum tolerated dose for administration of multiple injections of GNE-lipoplex in Balb/c mice was (1) 20 μg per injection (Human equivalent dose (HED)=5.2 mg), or (2) a cumulative dose of 80 μg (HED=20.8 mg). In the ongoing dose escalation trial, the patient has received several infusions (0.4, 0.4, 1.0 mg) of 1-3 months apart, and transient grade 1, 2 tachycardia and fever were observed within 12 hours of each infusion. Patient's liver function tests were also reported as transiently elevated, but exact numbers were not reported in the abstract (Nemunaitis, Jay et al. 2010).

Safety of Hydrodynamic Limb Vein (HLV) delivery method: Potential side effect of the hydrodynamic delivery method has been studied in non-human primates at double the tourniquet pressures proposed for the current study. The procedure was determined to be safe, without any non-reversible or long-lasting side effects (Vigen, Hegge et al. 2007; Hegge, Wooddell et al. 2010). Its procedure is similar to the Bier Block used for regional anesthesia and surgical homeostasis that has been used safely and effectively for over a century. The main difference is that exsanguination is unnecessary and duration of the procedure is typically 15 minutes in HLV (Hegge, Wooddell et al. 2010). Histologic studies in non-human primates have shown that the HLV procedure caused transient muscle edema but no significant muscle damage (Hagstrom, Hegge et al. 2004; Toumi, Hegge et al. 2006). T2-weighted MRI images in non-human primates also showed that the procedure caused transient muscle edema but there was no persistent muscle derangement such as a compartment syndrome (Vigen, Hegge et al. 2007). Magnetic resonance angiography in nonhuman primates revealed vascular effects consistent with a transient effect on capillary permeability but no long-term abnormalities of concern (Vigen et al., 2007). These initial studies were performed using much higher tourniquet pressures (700 mmHg) than we are proposing (310 mmHg). Also, the injection volume of 45-50% of the limb volume was used in these studies, and we are proposing an injection/limb volume of 35%. We believe the plasmid will enter myopathic fibers more effectively than normal muscle due to reduced integrity of the muscle cell walls, thus justifying the reduced pressures and injection volumes. Using these similar pressures, a volume escalation study in adult patients suffering from muscular dystrophy is underway at University of North Carolina, Chapel Hill (Powers, Fan et al. 2010).

In summary, the HLV delivery method using pDNA is considered mature technology that has proven effective and safe in non-human primates, and is ready to be tested in clinical therapeutic trials (Wells 2004; Al-Dosari, Knapp et al. 2005; Herweijer and Wolff 2007). The main disadvantage of this approach is the inability to easily transfect diaphragm, heart, and trunk/neck muscles without invasive methods to temporarily clamp the major internal vessels (e.g. surgical, laparoscopic, or transcutaneous balloon-occlusion). Although this disadvantage is significant for many muscular dystrophies, it is not nearly as important in patients affected by IBM2. Many IBM2 patients live into their senior years, their heart and lungs have not been reported to become clinically affected, trunk/neck muscles seem to remain strong until late in disease course, and there exists significant potential for bystander or distant effect. Thus, HLV delivery of pDNA for delivering GNE transgene to limb skeletal muscles is an attractive therapeutic option for IBM2 that may delay loss of physical independence, and offer significant hope for many IBM2 patients.

The GNE-encoding sequences and related compositions may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated composition or its delivery form. For example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent. Among the acceptable vehicles and solvents that may be employed are water, Ringer's solution, U. S. P. and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid may be used in the preparation of injectables.

According to certain embodiments, a Plasma-Lyte® carrier may be employed and used to deliver a GNE-encoding sequence, particularly for parenteral injection. (Baxter Laboratories, Inc., Morton Grove, Ill.). Plasma-Lyte® is a sterile, non-pyrogenic isotonic solution that may be used for intravenous administration. Each 100 mL volume contains 526 mg of Sodium Chloride, USP (NaCl); 502 mg of Sodium Gluconate (C6H11NaO7); 368 mg of Sodium Acetate Trihydrate, USP (C2H3NaO2\hat{ }H2O); 37 mg of Potassium Chloride, USP (KCI); and 30 mg of Magnesium Chloride, USP (MgCl2»6H2O). It contains no antimicrobial agents. The pH is preferably adjusted with sodium hydroxide to about 7.4 (6.5 to 8.0).

The injectable formulations used to deliver GNE-encoding sequences may be sterilized, for example, by filtration through a bacterial-retaining filter, or by incorporating sterilizing agents in the form of sterile solid compositions, which can be dissolved or dispersed in sterile water, Plasma-Lyte® or other sterile injectable medium prior to use.

In order to prolong the expression of a GNE-encoding sequence within a system (or to prolong the effect thereof), it may be desirable to slow the absorption of the composition from subcutaneous or intramuscular injection. This may be accomplished by the use of a liquid suspension of crystalline or amorphous material with poor water solubility. The rate of absorption of the composition may then depend upon its rate of dissolution, which, in turn, may depend upon crystal size and crystalline form.

Alternatively, delayed absorption of a parenterally administered GNE-encoding sequence may be accomplished by dissolving or suspending the composition in an oil vehicle. Injectable depot forms may be prepared by forming microencapsule matrices of the GNE-encoding sequence in biodegradable polymers such as polylactide-polyglycolide. Depending upon the ratio of GNE-encoding sequence material to polymer and the nature of the particular polymer employed, the rate of GNE-encoding sequence release can be controlled. Examples of other biodegradable polymers include poly(orthoesters) and poly(anhydrides). As described above, depot injectable formulations may also be prepared by entrapping the GNE-encoding sequence in liposomes (or even microemulsions) that are compatible with the target body tissues, such as muscular tissue.

In addition to methods for modulating the production of sialic acid in a system, the present invention further encompasses methods for producing wild-type GNE in a system. According to such embodiments, the system (e.g., the muscle cells of a human patient) may comprise a mutated endogenous GNE-encoding sequence (e.g., the GNE-M712T sequence). In other words, the present invention includes providing, for example, a cell or muscular tissue that harbors a mutated (defective) GNE-encoding sequence with a functional wild-type GNE encoding sequence. The wild-type GNE encoding sequence may be delivered to such a system using, for example, the liposomes or lipid nanoparticles described herein, via parenteral injection.

According to additional related embodiments of the present invention, methods for treating, preventing, and/or ameliorating the effects of Hereditary Inclusion Body Myopathy (HIBM2) are provided. Such methods generally comprise providing a patient with a therapeutically effective amount of a wild-type GNE-encoding nucleic acid sequence. In certain embodiments, the wild-type GNE-encoding nucleic acid sequence may, preferably, be delivered to a patient in connection with a lipid nanoparticle and a carrier similar to that of Plasma-Lyte®, via parenteral injection.

The phrase “therapeutically effective amount” of a wild-type GNE-encoding nucleic acid sequence refers to a sufficient amount of the sequence to express sufficient levels of wild-type GNE, at a reasonable benefit-to-risk ratio, to increase sialic acid production in the targeted cells and/or to otherwise treat, prevent, and/or ameliorate the effects of HIBM2 in a patient. It will be understood, however, that the total daily usage of the wild-type GNE-encoding nucleic acid sequence and related compositions of the present invention will be decided by the attending physician, within the scope of sound medical judgment.

One of the advantages of the methods described herein is that, because the polynucleotides are administered to the affected limb directly, as opposed to a systemic administration, the therapeutically effective amount that is administered is less than that in the methods described previously. Therefore, the present methods reduce or eliminate many of the side effects that are associated with the methods described previously.

The specific therapeutically effective dose level for any particular patient may depend upon a variety of factors, including the severity of a patient's HIBM2 disorder; the activity of the specific GNE-encoding sequence employed; the delivery vehicle employed; the age, body weight, general health, gender and diet of the patient; the time of administration, route of administration, and rate of excretion of the specific GNE-encoding sequence employed; the duration of the treatment; drugs used in combination or contemporaneously with the specific GNE-encoding sequence employed; and like factors well-known in the medical arts.

Upon improvement of a patient's condition, a maintenance dose of a GNE-encoding sequence may be administered, if necessary. Subsequently, the dosage or frequency of administration, or both, may be reduced, as a function of the symptoms, to a level at which the improved condition is retained when the symptoms have been alleviated to the desired level.

According to yet further embodiments of the invention, novel compositions are provided for expressing wild-type GNE in a system. The compositions preferably include a wild-type GNE-encoding nucleic acid sequence. As described herein, the GNE-encoding nucleic acid sequence may comprise various transcriptional control elements, such as a promoter, termination sequence, and others. A non-limiting example of a composition encompassed by the present invention includes the pUMVC3-GNE expression vector described herein, shown in FIG. 3. so as described relative to other embodiments of the present invention, the GNE-encoding nucleic acid sequence may be disposed within or connected to an appropriate vehicle for delivery to a system, such as a liposome or lipid nanoparticle. Still further, according to such embodiments, the delivery vehicle may, optionally, be decorated with agents that are capable of recognizing and binding to target cells or tissues, such as muscle cells or muscle tissues.

### EXAMPLES

### Example 1—Expression of Exogenous GNE in CHO-Lec3 Cells

In the following example, several GNE expression vectors from human cDNA were created. Three different GNE forms, wild type, M712T, and R266Q, were robustly expressed in GNE deficient cells (Lec3 cells). All enzymes demonstrated similar protein expression levels, albeit distinct enzymatic activities. As the following will show, the transfected GNE expressing cell lines produced significantly more sialic acid than untransfected cells.

### Methodology

### First Procedure

GNE Cloning. Parental vectors containing the GNE cDNA were provided by Daniel Darvish (HIBM Research Group, Encino, Calif.) and included pGNE-NB8 (wild type), pGNE-MB18 (M712T mutant), and pGNE-R266Q (R266Q mutant). The destination vector, pUMVC3, was purchased from Aldevron (Fargo, ND). The subcloning vector, pDrive (Qiagen, Valencia, Calif.)1 was used to shuttle the R266Q mutant from the parent vector to the destination vector.

GNE cDNA inserts (wildtype and M712T) were produced by reverse transcription of RNA isolated from patient whole blood. The R266Q isoform was produced using standard mutagenesis PCR techniques using specifically designed primers. cDNA was then amplified using specifically designed primers bearing EcoR1 and BamH1 recognition 5′ tails, and subsequently subcloned into the pUMVC3 expression vector (Aldevron) by T4 ligation (Invitrogen). Competent E. coli cells (Invitrogen) were then transformed with the pUMVC3 expression vector.

Positive pUMVC3-GNE clones were grown overnight in 175 mis LB broth+50 μg/ml Kan and 150 mis culture was used for a Qiagen (Valencia, Calif.) HiSpeed Plasmid Maxi kit according to the manufacturer protocols.

DNA.lipid complex. The DNA:lipid complex used in this example was produced by mixing, at room temperature, 1,2-Dioleoyl-3-Trimethylammonium-Propane (DOTAP) with test DNA (pUMVC3-GNE). DOTAP is a commercially-available lipid particle that is offered by Avanti Polar Lipids, Inc. (Alabaster, Alabama). The DOTAP was mixed with the pUMVC3-GNE DNA in a manner to achieve the desired total volume, which exhibited a final ratio of 0.5 μg DNA: 4 mM DOTAP1 in a final volume of 1 μl.

Cell Culture. GNE-deficient CHO-Lec3 cells were provided by Albert Einstein College of Medicine. The cells were grown at 37° C. in 5% CO₂ in α-MEM media supplemented with 4 mM L-glutamine and 10% heat inactivated, Fetal Bovine Serum. Cells for transient transfections were plated at 1×106 cells per well in 6-well plates and grown overnight. Lec3 cells were weaned to reduced serum conditions by reducing the FBS by 2.5% per passage.

Transient Transfections. Lec3 cells were transfected for 6 hours with DNA:lipid complex per well in OptiMEM (Invitrogen, Carlsbad Calif.), then the media was changed to normal α-MEM growth media and the cells were cultured overnight. DNA:lipid complexes were formed by mixing 4 μg DNA+10 μl Lipofectamine 2000 (Invitrogen) according to the manufacturers protocol. Twenty-four hours post-transfection, cells were harvested by trypsin digest and washed once with PBS before subsequent western blot or enzyme/sugar assays.

Sialic Acid Quantitation. Approximately 4×106 cells were used for the quantification of membrane-bound sialic acid by the thiobarbituric acid method. Cells were resuspended in water and lysed by passage through a 25 gauge needle 20 times and centrifuged. The supernatant was used for Bradford protein estimation and the remaining pellet was resuspended in 100 μl 2M acetic acid and incubated for 1 hour at 800 C to release glycoconjugate-bound sialic acids. 137 μl of periodic acid solution (2.5 mg/ml in 57 mM H₂SO₄) were added and incubated for 15 minutes at 37° C. Next, 50 μl of sodium arsenite solution (25 mg/ml in 0.5 M HCl) were added and the tubes were shaken vigorously to ensure complete elimination of the yellow-brown color. Following this step, 100 μl of 2-thiobarbituric acid solution (71 mg/ml adjusted to pH 9.0 with NaOH) were added and the samples were heated to 100° C. for 7.5 minutes. The solution was extracted with 1 ml of butanol/5% 12M HCl and the phases were separated by centrifugation. The absorbance of the organic phase was measured at 549 nm. The amount of sialic acid was measured as nmol sialic acid/mg of protein.

### Second Procedure

The following procedure is an alternative procedure to the one described above.

Cell culturing and biological assay testing: Lec3 CHO cells (Hong 2003) obtained from Dr. Pamela Stanley (Albert Einstein College of Medicine) were initially grown in α-MEM media containing 10% fetal bovine serum (FBS) (Invitrogen), received subsequent passages of α-MEM FBS medium by 2.5% decrements until 0% FBS, and trypsinized prior to transfection. Four sets of transfections were prepared in triplicate using 2.0×106 CHO cells, 2.5 mL of Freestyle Media (Invitrogen), 500 μl of Opti-MEM (Invitrogen), 10 μl of Lipofectamine (Invitrogen) and 4 μg of DNA (except for the no vector set) and incubated at 37° C. in 5% CO₂. Sets prepared included GNE wild-type pUMVC3 vector, GNE M712T pUMVC3 vector, GNE R266Q pUMVC3 vector, empty vector, and no vector media. Cells were collected 48 hours post-transfection, washed with PBS, and resuspended in lysis buffer. Sialic acid content was detected using a modified version of the Leonard Warren method (Warren 1959) and measured with NanoDrop-1000 Spectrophotometer (Thermo Fisher Scientific) at 549 nm using the UBV-Vis module. A standard curve was created with known sialic acid concentrations and denoted a clear linear association between absorbance and sialic acid concentration.

### Results

GNE clones. The GNE cDNA clones that were tested included a human wild type cDNA and two human mutant cDNAs. The mutants included the M712T GNE deficient clone and the R266Q sialuria clone. Sialuria is a human disease caused by point mutations in the CMP-sialic acid binding site of GNE, leading to a loss of feedback inhibition and mass production of sialic acids. GNE cDNAs were subcloned from their original vectors to the expression vector, pUMVC3, by restriction digest cloning. Clones were screened by directional restriction enzyme digest to confirm the GNE insert was in the correct orientation. Positive clones were sequenced in both orientations to confirm that no mutations occurred during the cloning process. The resulting chromatograms were compared against the GNE sequence from GenBank (accession #NM_005467) and the wild type did not exhibit any mutations, while the M712T and R266Q clones contained only the expected point mutations. Positive pUMVC3-GNE clones were scaled using a maxi prep plasmid purification procedure and sequenced again to confirm that no mutations occurred. These DNA stocks were used for all subsequent experiments.

Wt-GNE mRNA quantitation. CHO-Lec3 cells were grown in 10% serum and transiently transfected with pUMVC3-GNE-wt DNA for 24 hours to quantitate the amount of recombinant GNE RNA that was expressed. Total RNA was extracted and RT-qPCR was performed to amplify a 230 bp fragment from the GNE transcript. Serial dilutions of pUMVC3-GNE-wt were used to determine that the concentration of GNE-wt expressed in transfected Lec3 cells was equal to 4.1 pg/μl. The dynamic range of the qPCR was from 5 ng-5 fg and there was no GNE mRNA product detected in control (untransfected) CHO-Lec3 cells (the cT value for untransfected cells was greater than 42 cycles, which is less than 5 fg). Therefore, recombinant GNE mRNA expression was detected in transfected Lec3 cells, while untransfected cells had undetectable amounts of GNE mRNA.

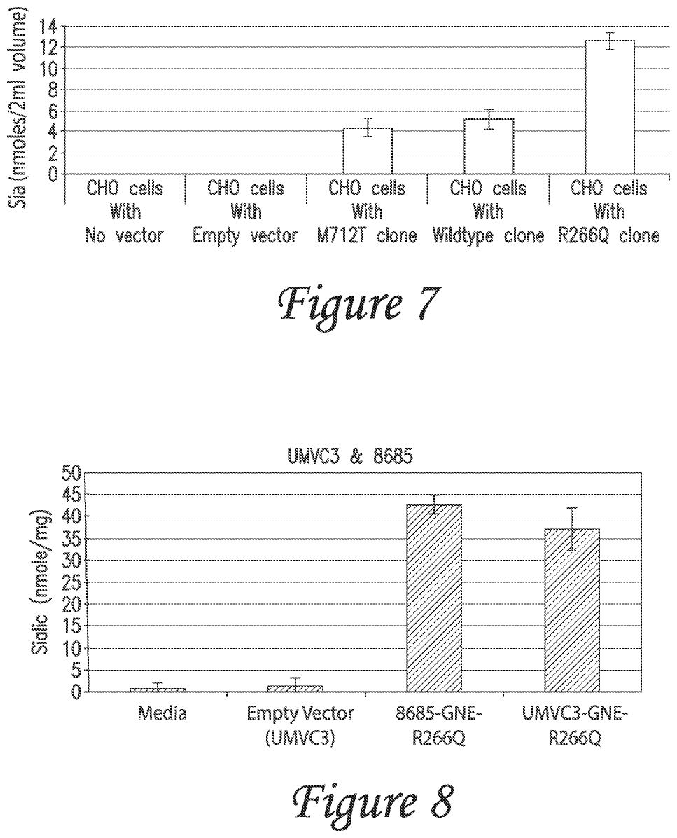

Sialic acid assays. Transfected Lec3 cells also were tested for cell surface sialic acid expression. All Lec3 samples had approximately 6.0 nmol/mg membrane bound sialic acid, with the exception of Lec3 cells transfected with the R266Q GNE1 which had a 1.5-fold higher amount (FIG. 7). The R266Q GNE lacks the feedback inhibition of GNE and is known to cause an overproduction of intracellular sialic acids. Lec3 cells seem to be undersialylated, and this could only be overcome by expression of the sialuria mutant and not by the about 100-fold overexpression of wild-type GNE compared to wild-type CHO cells. No significant differences between wild type (wt) and M712T GNE were observed.

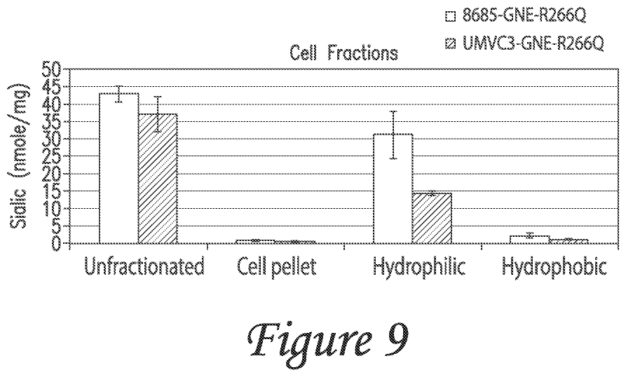

Comparison of UMVC3 and NTC8685 GNE plasmids: Transfection studies comparing sialic acid production of both vectors correlated well with each other (FIGS. 8 and 9). Slightly higher production of sialic acid was noted with NTC8685 vector. Additional in-vitro studies using other cell types and in-vivo studies will be conducted.

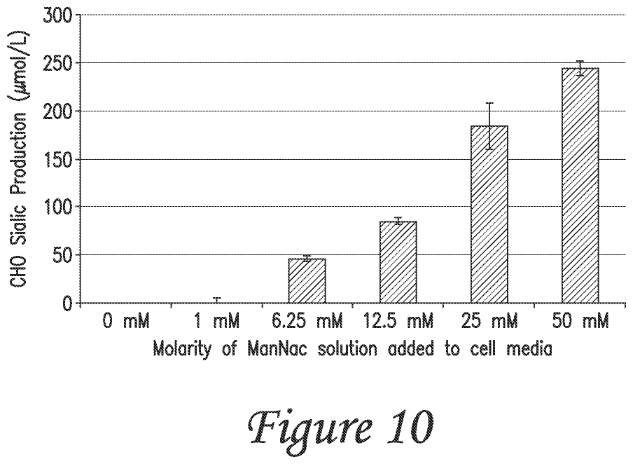

Silic acid production by provision of ManNAc. The level of Sialic acid production was measured by supplementing cell culture media with N-Acetylmannosamine (ManNAc). Besides provision of ManNAc, all other cell culture variables were identical to transfection studies (FIG. 10).

Preliminary high dose plasmid toxicity. We conducted a recent pre-GLP toxicology study of 14 day duration on 12 mice (strain B6; FBV mixed inbred, 6 male and 6 female of age 4-10 months) . Male and female mice were divided equally and randomly into experiment and control groups (Table 1). The maximum feasible dose (MFD) in a mouse model was 600 μg per injection. Limitation was based on solubility of plasmid (6 μg/μl) and total volume per injection (100 μL). Considering mouse weight of 30 g and human weight of 70 kg, the human equivalent dose (HED) for mouse dose of 600 μg is 113.82 mg.

TABLE 1

| | | | | | | | | | | Total |
|---|---|---|---|---|---|---|---|---|---|---|
| | Frequency | | Weight (g) | Toxicity | Toxicity | Toxicity | Weight | Toxicity | Weight | Plasmid |
| | of infusion | Mice | Day 1 | 24 h | 48 hr | Day 7 | Day 7 | Day 14 | Day 14 | Dose |
| Control Group | Every day | 1M | 29.54 | None | None | None | 28.8 | None | 28.96 | 0 |
| (100 normal | | 1F | 29.99 | None | None | None | 26.6 | None | 26.74 | 0 |
| saline) | Every other | 1M | 32.69 | None | None | None | 32.9 | None | 31.95 | 0 |
| | day | 1F | 21.88 | None | None | None | 20.6 | None | 20.23 | 0 |
| | Once per | 1M | 27.76 | None | None | None | 27.5 | None | 26.91 | 0 |
| | week (day 1 | 1F | 22.24 | None | None | None | 22.5 | None | 23.55 | 0 |
| | and 7) | | | | | | | | | |
| Experiment | Every day | 1M | 27.59 | None | None | None | 26.8 | None | 27.68 | 8.4 mg |
| Group (600 ug | | 1F | 27.28 | None | None | None | 24.7 | None | 21.78 | 8.4 mg |
| plasmid in 100 | Every other | 1M | 31.54 | None | None | None | 29.6 | None | 29.39 | 4.2 mg |
| uL NS) | day | 1F | 23.35 | None | None | None | 21.9 | None | 23.71 | 4.2 mg |
| | Once per | 1M | 30.37 | None | None | None | 28 | None | 29.8 | 1.2 mg |
| | week (day 1 | 1F | 24.55 | None | None | None | 23 | None | 23.38 | 1.2 mg |
| | and 7) | | | | | | | | | |

The experiment group received high dose GNE plasmid (0.6 mg suspended in 0.1 ml normal saline) administered via IV by tail vein, and the control group received 0.1 ml normal saline. The groups were further divided into 3 dose frequency groups of 2 mice (lfemale, 1 male) each as follows: 1) Every day administration for 14 days, 2) Every other day administration, and 3) Once per week. All animals survived the experiment. No significant change were observed between the experiment and the control groups with respect to all measured parameters, which included body weights, temperature, food and water intake, CBC blood tests (performed at days 1 and 15). Following necropsy on day 15, no significant change in the gross pathology was observed between the experiment and the control groups with respect to 12 organs, including brain, lung, heart, liver, kidney, spleen, stomach, intestines, bladder, genitals, lymph nodes, and muscle.

Although illustrative embodiments of the present invention have been described herein, it should be understood that the invention is not limited to those described, and that various other changes or modifications may be made by one skilled in the art without departing from the scope or spirit of the invention.

What is claimed is:

1. A method of treating hyposialylation in a subject, comprising administering to the subject a composition comprising a net negative charge, and a DNA molecule encoding GNE or a therapeutic fragment thereof.

2. The method of claim 1, wherein the composition comprises hypermorphic GNE.

3. The method of claim 1, wherein the composition is administered by hydrodynamic delivery route.

4. The method of claim 1, wherein the composition is a gene therapy vector or an enzyme replacement therapy.

5. The method of claim 1, wherein GNE has at least one mutation within the allosteric domain.

6. The method of claim 1, wherein subsequent to the administration of the composition, the subject experiences an increase in sialic content.

7. The method of claim 1, wherein the subject has at least one mutation in the gene encoding GNE.

8. The method of claim 1, comprising administering the composition to a limb or limbs of the subject.

9. The method of claim 1, wherein subsequent to the administration of the composition, the subject experiences an improvement in hyposialylation.

10. The method of claim 1, wherein subsequent to the administration of the composition, the subject experiences an improvement in the muscle function.

11. The method of claim 1, wherein the subject suffers from muscle wasting disorder.

12. The method of claim 1, wherein the subject is diagnosed with GNE related myopathy.
